(19) United States
(12) Patent Application Publication
(10) Pub. No.: US 2025/0002547 A1
(43) Pub. Date: Jan. 2, 2025

(54) MUTANT TDP-43 PROTEIN

(71) Applicant: TOKYO METROPOLITAN INSTITUTE OF MEDICAL SCIENCE, Tokyo (JP)

(72) Inventors: Takashi NONAKA, Tokyo (JP); Mina TAKASE, Tokyo (JP); Masato HASEGAWA, Tokyo (JP)

(73) Assignee: TOKYO METROPOLITAN INSTITUTE OF MEDICAL SCIENCE, Tokyo (JP)

(21) Appl. No.: 18/754,639

(22) Filed: Jun. 26, 2024

(30) Foreign Application Priority Data
Jun. 30, 2023 (JP) 2023-108532

(51) Int. Cl. C07K 14/47; A61K 49/00; G01N 33/50

(52) U.S. Cl. CPC C07K 14/4703; A61K 49/0008; G01N 33/5058; G01N 33/5091

(57) ABSTRACT

A mutant TDP-43 protein, having a deletion of a nuclear localization signal sequence in the amino acid sequence of a wild-type TDP-43 protein, and also having any one of the following mutations (a) to (c) or a combination of these mutations:
(a) a mutation, in which the 147th and 149th phenylalanines are substituted with leucines,
(b) a mutation, in which the 194th phenylalanine is substituted with leucine, and
(c) a mutation, in which the 229th and 231st phenylalanines are substituted with leucines.

### REFERENCE TO ELECTRONIC SEQUENCE LISTING

The application contains a Sequence Listing which has been submitted electronically in .XML format and is hereby incorporated by reference in its entirety. Said .XML copy, created on Jun. 26, 2024, is named “G3389US_SeqListing.xml” and is 25,267 bytes in size. The sequence listing contained in this .XML file is part of the specification and is hereby incorporated by reference herein in its entirety.

### BACKGROUND OF THE INVENTION

### Field of the Invention

The present invention relates to a mutant TDP-43 protein, and a cell model and a mouse model for mutant TDP-43 accumulation, comprising the mutant TDP-43 protein.

### Description of the Related Art

In the brains and the like of many patients with neurodegenerative diseases, intracellular aggregates that characterize the diseases appear in neurons and glial cells. The major constituent proteins of intracellular aggregates are different depending on each disease, and Tau in Alzheimer's disease (AD), α-synuclein in Parkinson's disease (PD) and dementia with Lewy bodies (DLB), and TDP-43 in amyotrophic lateral sclerosis (ALS) and frontotemporal lobar degeneration (FTLD) have been identified as major constituent proteins of intracellular aggregates specific to individual diseases. However, the mechanism of aggregation of specific proteins in cells has hardly been clarified. Thus, for the purpose of elucidating the mechanism, various models have been constructed to reproduce the intracellular accumulation of Tau, α-synuclein and TDP-43, but only a few models faithfully reproduce the protein accumulation pathology in the patient brain. Regarding TDP-43, since it has been reported in 2006 that TDP-43 is a major constituent protein of ubiquitin-positive intracellular aggregates in the brains of FTLD patients (Arai et al, 2006, and Neuman et al, 2006), a large number of TDP-43 expressing mice have been developed (Wils et al, 2010). In addition, cell models for TDP-43 accumulation have also been studied (Japanese Patent No. 5667872).

However, a model that fully reproduces the intracellular accumulation of phosphorylated TDP-43 in the patient brain has not been reported so far, and its development is still awaited.

### CITATION LIST

### Patent Literature

Patent Literature 1: Japanese Patent No. 5667872

### Non Patent Literature

Non Patent Literature 1: Arai et al, Biochem Biophys Res Commun. 2006 Dec. 22; 351 (3): 602-11.
Non Patent Literature 2: Neumann et al, Science. 2006 Oct. 6; 314 (5796): 130-3.
Non Patent Literature 3: Wils H et al, Proc Natl Acad Sci USA. 2010 Feb. 23; 107 (8): 3858-63.

Against the aforementioned background, it has been desired to develop a model that fully reproduces the intracellular accumulation of phosphorylated TDP-43 observed in the patient brain.

### SUMMARY OF THE INVENTION

As a result of intensive studies directed towards achieving the aforementioned object, the present inventor has found that the aforementioned object can be achieved by deleting the nuclear localization signal of wild-type TDP-43 and substituting some amino acids in the RNA-binding domain with other amino acids.

Specifically, the present invention is as follows.

[1] A mutant TDP-43 protein, having a deletion of a nuclear localization signal sequence in the amino acid sequence of a wild-type TDP-43 protein, and also having any one of the following mutations (a1) to (c2) or a combination of these mutations, wherein
the mutant TDP-43 protein has aggregation activity in cells:
(a1) a mutation, in which the 147th and 149th phenylalanines are substituted with leucines,
(a2) a mutation, in which the 147th and 149th phenylalanines are substituted with leucines, and one or several amino acids other than the 147th and 149th amino acids are deleted, substituted, or added,
(b1) a mutation, in which the 194th phenylalanine is substituted with leucine,
(b2) a mutation, in which the 194th phenylalanine is substituted with leucine, and one or several amino acids other than the 194th amino acid are deleted, substituted, or added,
(c1) a mutation, in which the 229th and 231st phenylalanines are substituted with leucines, and
(c2) a mutation, in which the 229th and 231st phenylalanines are substituted with leucines, and one or several amino acids other than the 229th and 231st amino acids are deleted, substituted, or added.
   [2] The mutant TDP-43 protein according to the above [1], wherein the amino acid sequence of the wild-type TDP-43 protein is as set forth in SEQ ID No: 2, 4, 6 or 8.
   [3] DNA encoding the mutant TDP-43 protein according to the above [1].
   [4] A vector comprising the DNA according to the above [3].
   [5] The vector according to the above [4], which is an adeno-associated virus vector.
   [6] An animal model for mutant TDP-43 protein accumulation, in which the mutant TDP-43 protein according to the above [1] is expressed in the brain of a non-human mammal.
   [7] A cell model for mutant TDP-43 protein accumulation, in which an aggregate of the mutant TDP-43 protein according to the above [1] is introduced into a cell.
   [8] The cell model according to the above [7], wherein the aggregate of the mutant TDP-43 protein is derived from the brain of the animal model according to the above [6.
   [9] The cell model according to the above [8], wherein the aggregate of the mutant TDP-43 protein has seeding activity by which it functions as a seed for intracellular accumulation of the protein.

A method of screening for a therapeutic drug for neurodegenerative disease, which is characterized in that it comprises contacting or administering a test candidate substance to the animal model according to the above [6] or the cell model according to the above [7].

A kit of screening for a therapeutic drug for neurodegenerative disease, including the animal model according to the above [6] or the cell model according to the above [7].

### Advantageous Effects of the Invention

According to the present invention, a mutant TDP-43 protein is provided. A cell or a mouse, into which the mutant protein of the present invention is introduced, is useful as a cell model or an animal model for neurodegenerative disease.

### BRIEF DESCRIPTION OF THE DRAWINGS

FIG. 1 is a view showing mutation introduction positions in the amino acid sequence of a mutant TDP-43 protein, which has no binding ability with nucleic acids.

FIG. 2 shows fluorescence microscopic images of TDP-43 FL mutant-expressing cells.

FIG. 3-1 is a view showing the results of immunoblot analysis of TDP-43 FL mutant-expressing cells.

Plasmid-expressing cells were homogenized in the presence of the detergent sarkosyl, and the soluble fraction (Sar-sup) and the insoluble (Sar-ppt) fraction thereof were analyzed. Intracellularly accumulated, phosphorylated TDP-43 (red arrows) was detected in the Sar-ppt fraction by a phosphorylated TDP-43-specific antibody (anti-pS409/410).

FIG. 3-2 is a view showing the results of immunoblot analysis of TDP-43 FL mutant-expressing cells.

Plasmid-expressing cells were homogenized in the presence of the detergent sarkosyl, and the soluble fraction (Sar-sup) and the insoluble (Sar-ppt) fraction were analyzed. Intracellularly accumulated, phosphorylated TDP-43 (red arrows) was detected in the Sar-ppt fraction by a phosphorylated TDP-43-specific antibody (anti-pS409/410).

FIG. 4 is a view showing an outline of a mouse model experiment.

FIG. 5 is a view showing the expression of TDP-43ΔNLS&FL-A in mouse brain by TDP-43ΔNLS&FL-A expressing AAV.

AAV that expresses TDP-43ΔNLS&FL-A specifically in neurons was inoculated and infected into adult mouse brains. One month after the AAV inoculation, the brains were excised and were subjected to immunoblot analysis. The collected right mouse brains were homogenized using one type of detergent, sarcosyl, and were then centrifuged to obtain sarcosyl-soluble (Sar-sup) fractions. These were immunoblotted using human TDP-43 specific antibodies. Red arrows: TDP-43ΔNLS&FL-A.

In mice infected with 1×10⁹ vg of AAV (Lanes 1 to 5), the expression of TDP-43ΔNLS&FL-A of interest was observed, while the expression level was reduced in mice infected with 1×10⁸ vg of AAV (Lanes 6 to 10). In mice inoculated with a normal saline (Lane 11), the expression could not be confirmed.
1 to 5: mice infected with 1×10⁹ vg of AAV (total 5 mice)
6 to 10: mice infected with 1×10⁸ vg AAV (total 5 mice)
11: Mice inoculated with a normal saline

FIG. 6 is a view showing the immunoblot analysis of mouse brain in which TDP-43ΔNLS&FL-A was expressed.

AAV that expresses TDP-43ΔNLS&FL-A specifically in neurons was inoculated and infected into adult mouse brains. One month after the AAV inoculation, the brains were excised and were subjected to immunoblot analysis. The collected right mouse brains were homogenized using one type of detergent, sarcosyl, and were then centrifuged to obtain sarcosyl-insoluble (Sar-ppt) fractions. These were immunoblotted using phosphorylated TDP-43 antibodies. Red arrow: TDP-43ΔNLS&FL-A.

In mice infected with 1×10⁹ vg of AAV, insoluble TDP-43ΔNLS&FL-A of interest was observed in the brain (although there were individual differences). On the other hand, in mice infected with 1×10⁸ vg of AAV or a normal saline, accumulation of TDP-43 was not observed.
1 to 5: mice infected with 1×10⁹ vg of AAV (total 5 mice)
6 to 10: mice infected with 1×10⁸ vg AAV (total 5 mice)
11: Mice inoculated with a normal saline

FIG. 7 is a view showing the immunostaining of mouse brains expressing TDP-43ΔNLS&FL-A.

AAV (1×10⁹ vg) that expresses TDP-43ΔNLS&FL-A specifically in neurons was inoculated and infected into adult mouse brains. One month after the AAV inoculation, the brains were excised, and the left brains were fixed in formalin and were then subjected to immunohistochemical staining using human TDP-43-specific antibodies. The left brain was analyzed by immunohistochemical staining. As a result, TDP-43 antibody-positive staining images (brown color) were obtained in the neurons of the hippocampus and the medial posterior parietal cortex.

FIG. 8 is a view showing the formation of an aggregate of phosphorylated TDP-43 in TDP-43 ΔNLS&FL-A-expressing mouse brains.

AAV (1×10⁹ vg) that expresses TDP-43ΔNLS&FL-A specifically in neurons was inoculated and infected into adult mouse brains. One month after the AAV inoculation, the brains were excised, and the left brains were then used in immunohistochemical staining using phosphorylated TDP-43-specific antibodies. As a result, intracellular aggregates positive for phosphorylated TDP-43 antibodies (dark brown staining images) were observed in the neurons of the hippocampus and the medial posterior parietal cortex. Such staining images were not observed in normal saline-ingested mouse brains.

FIG. 9 is a view showing the seeding activity of a sarcosyl-insoluble fraction of AAV-infected mouse brain using cultured cells.

Insolubilized TDP-43 obtained from mouse brains infected with AAV expressing TDP-43ΔNLS&FL-A was added as a seed to SH-SY5Y cells transiently expressing TDP-43ΔNLS plasmid. After the cells were collected, the recovered cells were homogenized with one type of detergent, sarcosyl, and were then centrifuged to obtain sarcosyl-insoluble (Sar-ppt) fractions. These were immunoblotted using phosphorylated TDP-43 antibodies. Red arrow: phosphorylated TDP-43
1 and 2: Untreated
3: TDP-43 ANLS plasmid expression only
4: TDP-43ΔNLS plasmid expression+insolubilized TDP-43 in mouse brain 1 month after AAV infection
5: TDP-43ΔNLS plasmid expression+insolubilized TDP-43 in mouse brain 3 months after AAV infection
6: TDP-43ΔNLS plasmid expression+insolubilized TDP-43 prepared from ALS patient brain

### DETAILED DESCRIPTION OF THE PREFERRED EMBODIMENTS

### 1. Outline

The present invention relates to a mutant TDP-43 protein, having a deletion of a nuclear localization signal sequence in the amino acid sequence of a wild-type TDP-43 protein, and also having any one of the following mutations (a1) to (c2) or a combination of these mutations, wherein
the mutant TDP-43 protein has aggregation activity in cells:
(a1) a mutation, in which the 147th and 149th phenylalanines are substituted with leucines,
(a2) a mutation, in which the 147th and 149th phenylalanines are substituted with leucines, and one or several amino acids other than the 147th and 149th amino acids are deleted, substituted, or added,
(b1) a mutation, in which the 194th phenylalanine is substituted with leucine,
(b2) a mutation, in which the 194th phenylalanine is substituted with leucine, and one or several amino acids other than the 194th amino acid are deleted, substituted, or added,
(c1) a mutation, in which the 229th and 231st phenylalanines are substituted with leucines, and
(c2) a mutation, in which the 229th and 231st phenylalanines are substituted with leucines, and one or several amino acids other than the 229th and 231st amino acids are deleted, substituted, or added.

The present inventors have found that when mutant TDP-43, which cannot bind to RNA, is expressed in cultured cells, it is phosphorylated and accumulates in the cells. Furthermore, when adeno-associated virus (AAV) encoding this mutant TDP-43 was prepared and was then inoculated into mouse brain, phosphorylated TDP-43-positive intracellular aggregates were observed in the brain. This novel mutant TDP-43-expressing mouse model is considered to be useful not only for elucidating the mechanism of intracellular TDP-43 accumulation, but also for developing therapeutic drugs and/or preventive methods for ALS, FTLD and other diseases.

In amyotrophic lateral sclerosis (ALS) and some frontotemporal lobar degeneration (FTLD), intracellular aggregates consisting of TDP-43 are found in neurons and glial cells. TDP-43 is one type of RNA-binding protein that localizes in the nucleus, and in the brain of patients, TDP-43 accumulates in the cytoplasm and the nucleus through post-translational modifications such as phosphorylation and ubiquitination. To date, many genetically engineered mice and the like have been produced to elucidate the mechanism of TDP-43 accumulation. However, a model that fully reproduces the TDP-43 pathology observed in the brains of ALS and FTLD patients has not yet been developed.

In the present invention, the object of the invention is to develop a model that faithfully reproduces the abnormal lesions of TDP-43 that appear in the brains, etc. of patients by allowing mutant TDP-43 to express in mouse brains, using an adeno-associated virus (AAV) expression system. AAV expressing mutant TDP-43 was inoculated into mouse brains, and the brains were then excised several months later, which were then subjected to immunohistochemical staining and biochemical analysis. As a result, phosphorylated TDP-43 antibody-positive intracellular aggregates were observed in the brains, and insolubilization of phosphorylated TDP-43 was biochemically confirmed.

When insolubilized TDP-43 aggregates prepared from these mouse brains were introduced into TDP-43-expressing cultured cells, the insolubilized TDP-43 aggregates functioned as seeds, and accumulation of TDP-43 in the cultured cells was enhanced. A mouse with phosphorylated TDP-43 accumulating in the brain thereof, which was constructed in the present studies, can be used to elucidate the mechanism of intracellular accumulation of TDP-43 and to develop therapeutic drugs such as compounds that inhibit TDP-43 accumulation. In addition, since insolubilized TDP-43 prepared from this mouse brain functions as a seed to induce its accumulation, it can be utilized to construct a cellular model and the like, involving TDP-43 accumulation, and is considered to be useful as a novel tool that replaces for conventional seeds derived from patient brains.

For the purpose of investigating the relationship between the RNA-binding ability and the aggregation properties of TDP-43, the present inventors have prepared several mutants of TDP-43, in which mutations are introduced into its RNA-binding domain, and have examined the effects of the mutants.

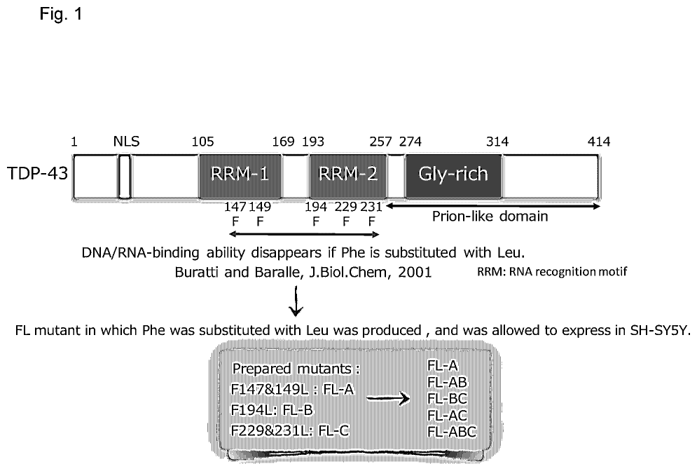

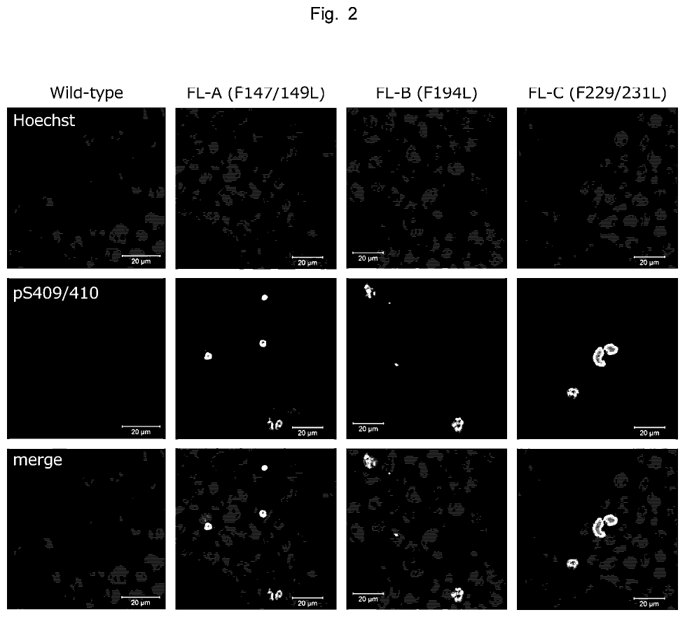

Regarding TDP-43, it has been reported that the nucleic acid-binding ability of TDP-43 disappears if several Phe residues existing in the RNA-binding domain are substituted with Leu residues (FIG. 1, Buratti and Baralle, 2001). Thus, mutants (FL mutants, FIG. 1), in which several Phe residues were substituted with Leu residues, were produced, and the mutants were transiently expressed in cultured cells. As a result, phosphorylated TDP-43 antibody-positive intracellular aggregates were most abundantly observed in cells expressing the mutant (FL-A), in which the 147th and 149th Phe residues were substituted with Leu residues (FIG. 2).

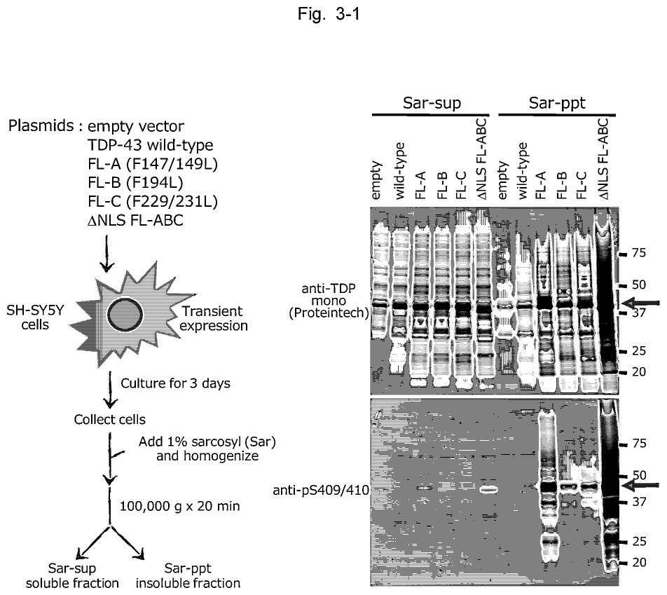

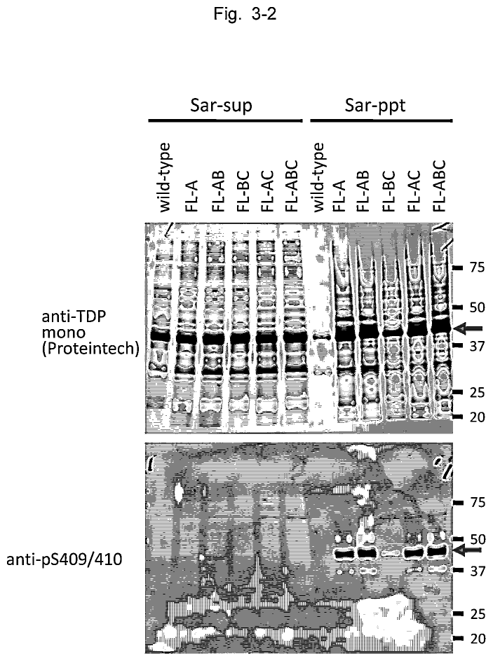

Furthermore, in the biochemical analysis of FL mutant-expressing cell lysates (FIG. 3), a phosphorylated TDP-43 antibody-positive band was observed in the detergent sarkosyl (Sar)-insoluble fraction (Sar-ppt) in FL-A mutant-expressing cells, and it was confirmed that this mutant aggregates in the cells. In addition, in mutant (ΔNLS&FL-ABC) expressing cells having a mutation (ANLS) of deletion of the nuclear localization signal (NLS) as well as the FL mutation, significant intracellular accumulation of phosphorylated TDP-43 was observed, compared with the cells expressing the FL-A mutant. Moreover, it was found from FIG. 3-2 that the effects of each FL mutation on aggregation properties depend only on FL-A, while the mutation effects of FL-B and C are very low.

From the aforementioned results, it became clear that when the FL mutant, which is involved in the nucleic acid-binding ability of TDP-43, is expressed in cultured cells, accumulation of phosphorylated TDP-43 occurs in the cells. Moreover, considering that the intracellular accumulation of TDP-43 was most abundantly observed in FL-A among the FL mutants (FIG. 1), and that the accumulation of TDP-43 was significantly increased by introducing a mutation involving deletion of NLS into the cells (FIG. 3), a new mutant, TDP-43 ΔNLS&FL-A, was prepared by introduction of these two mutations. Thereafter, whether the intracellular accumulation of phosphorylated TDP-43 occurs even in mouse brain by allowing the aforementioned new mutant to express in the mouse brain was examined.

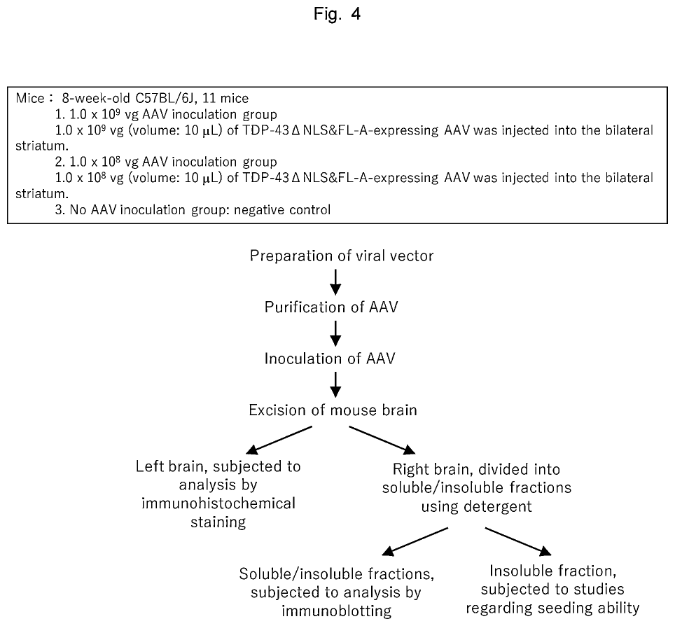

AAV virus (serotype: AAV-PHP.eB) expressing TDP-43 ΔNLS&FL-A was prepared using HEK293 cells according to an ordinary method. The medium of the plasmid-transfected cells was collected, a virus solution was concentrated by an ultrafiltration method, and its concentration was measured by a real-time PCR method. 1.0×10⁸ vg or 1.0×10⁹ vg of the obtained virus was inoculated into the bilateral striatum of adult mouse brain. One month or three months after the inoculation of the virus, the brain was excised, and then, the right brain was subjected to biochemical analysis (immunoblotting) and the left brain to immunohistochemical analysis (FIG. 4).

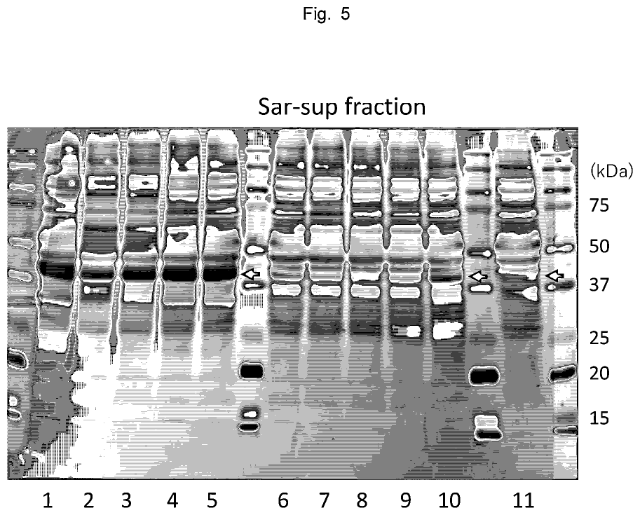

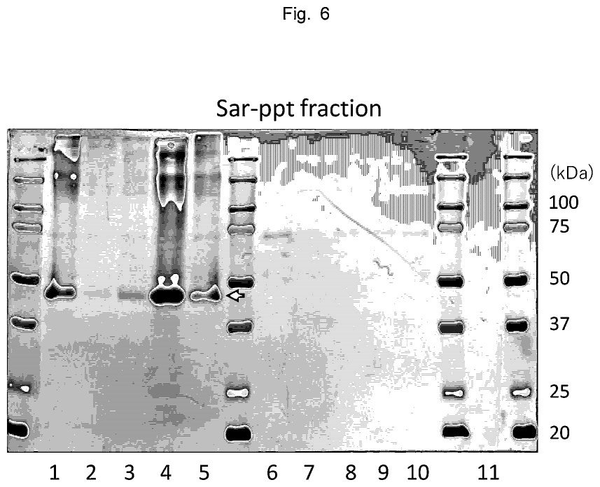

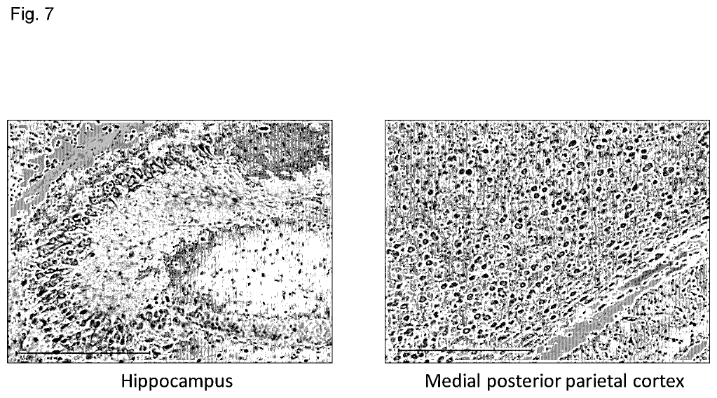

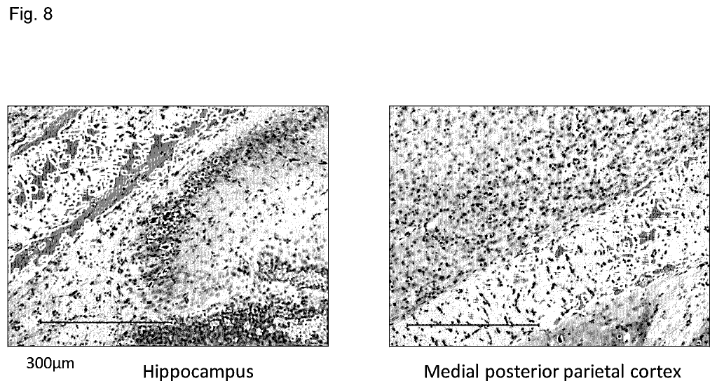

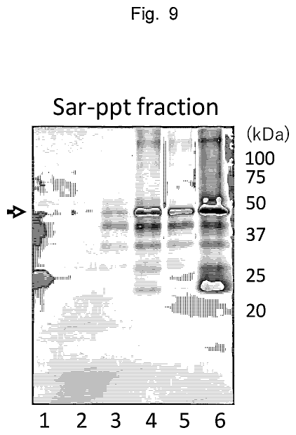

One month after the infection, the mouse brain (right brain) was homogenized in a buffer containing sarcosyl, and was then centrifuged. The obtained supernatant (soluble fraction: Sar-sup) and precipitate (insoluble fraction: Sar-ppt) were collected and were subjected to immunoblot analysis. According to the analysis of the Sar-sup fraction shown in FIG. 5, the expression of TDP-43 ΔNLS&FL-A of interest was observed in virus-infected mice (the red arrow bands in FIG. 5). The expression level was dependent on the amount of the virus inoculated into the mouse brain, and a higher expression was observed in the mouse brain infected with 1.0×10⁹ vg of AAV. On the other hand, the expression was not observed in mice inoculated with a normal saline instead of the virus.

Subsequently, in the results of the immunoblotting of the Sar-ppt fraction shown in FIG. 6, a phosphorylated TDP-43-specific antibody-positive band was observed in the sarcosyl-insoluble fraction of mouse brain infected with 1.0×10⁹ vg of AAV (the red arrow). The intensity of the band varied among individuals, but these bands were not detected in mouse brains infected with a normal saline or 1.0×10⁸ vg of AAV. From these results, it became clear that TDP-43 ΔNLS&FL-A expressed in brain by AAV infection is phosphorylated and aggregated in the cells.

From the aforementioned results of the biochemical analysis of AAV-infected mouse brains, it was found that the expression of TDP-43 ΔNLS&FL-A was significantly observed in mice infected with 1.0×10⁹ vg of the virus. Thus, the immunohistochemical analysis of these mouse brains was conducted. One month after infection with 1.0×10⁹ vg of AAV, the mouse brains were excised, and their left brains were fixed in formalin. Thereafter, paraffin blocks were prepared, and ultrathin slices (i.e. 8-μm thick slices) were prepared and stained with human TDP-43-specific antibodies and phosphorylated TDP-43-specific antibodies.

As a result, as shown in FIG. 7, the expression of TDP-43 ΔNLS&FL-A was observed in the hippocampus, the medial posterior parietal cortex, and other regions by the AAV virus infection. In addition, the sections of the AAV-infected mouse brains were stained using phosphorylated TDP-43-specific antibodies specifically reacting with phosphorylated TDP-43 aggregates appearing in the patient brains. As a result, the phosphorylated TDP-43-specific antibody-positive staining images were observed in the hippocampus, the medial posterior parietal cortex, and other regions (FIG. 8).

From these results, it became clear that phosphorylated TDP-43 ΔNLS&FL-A forms intracellular aggregates in this AAV-infected mouse brain, and that a novel mouse model that faithfully reflects the TDP-43 pathology of the patient brain could be constructed.

TDP-43 accumulated in the brain of ALS or FTLD patients has been reported to have prion-like properties (Nonaka et al, 2013). That is, when insolubilized TDP-43 prepared as a sarcosyl-insoluble fraction from the patient brain is introduced into cultured cells expressing TDP-43 NLS, it functions as a seed to induce aggregation of TDP-43 ANLS TDP-43 ANLS in the cells (FIG. 9; Lane 6). Thus, whether TDP-43 ΔNLS&FL-A aggregated in the AAV-infected mouse brain also has similar prion-like properties, namely, whether it can function as a seed, was examined.

50 μL of a normal saline was added to sarcosyl-insoluble fractions (Lane 4, etc. of FIG. 6) prepared from AAV-infected mouse brains, and the resulting factions were then subjected to an ultrasonic treatment. The thus obtained fractions were used as insolubilized TDP-43 ΔNLS& FL-A. To SH-SY5Y cells that had previously expressed TDP-43 ΔNLS, 5 μL of insolubilized TDP-43 ΔNLS& FL-A was added together with MultiFectam reagent (Promega). After incubation for several days, the cells were collected, were homogenized in a sarcosyl-containing buffer, and were centrifuged to obtain a sarcosyl-insoluble fraction (Sar-ppt). The Sar-ppt was subjected to immunoblot analysis using a phosphorylated TDP-43-specific antibody. Even if TDP-43 ANLS was allowed to express alone in cultured cells, it did not accumulate in the cells, and therefore almost no bands were observed in the Sar-ppt fraction (FIG. 9; Lane 3).

On the other hand, when insolubilized TDP-43 prepared from ALS patient brains is added as a seed to cells, it functions as a seed and induces accumulation of TDP-43 ANLS in the cells (FIG. 9; Lane 6). Similarly, when insolubilized TDP-43 ΔNLS&FL-A prepared from AAV-infected mouse brains was added to cells, phosphorylated TDP-43-specific antibody-positive bands appeared in the Sar-ppt fraction (FIG. 9; Lanes 4 and 5). Thus, it became clear that the insolubilized TDP-43 ΔNLS&FL-A observed in the AAV-infected mouse brains has prion-like properties, as with the insolubilized TDP-43 observed in the patient brains.

Meanwhile, as shown in FIG. 3, when TDP-43 FL-A or ΔNLS&FL mutants are allowed to transiently express in cultured cells, those mutant TDP-43 accumulate in the cells. The applicants also examined whether insolubilized TDP-43 could function as a seed in cultured cells, but the insolubilized TDP-43 did not function as a seed. The mutant TDP-43 was almost identical to the type expressed in mouse brains, but only the insolubilized TDP-43 accumulated in mouse brains had seeding activity.

Until now, the seed that can efficiently induce the intracellular accumulation of TDP-43 in, for example, cultured cells, is only the insolubilized TDP-43 derived from patient brains, and its use has been limited. In addition, the use of patient brain raises ethical issues, and therefore, it is not available to many researchers, and thus, a tool as a research reagent that can be easily used by everyone has been desired.

In TDP-43 ΔNLS&FL-A-expressing mice found in the present invention, it has been revealed that the TDP-43 ΔNLS&FL-A does not only accumulate in the mouse brain in such an early period as one month after AAV infection, but also the insolubilized TDP-43 ΔNLS&FL-A has prion-like activity similar to that of the insolubilized TDP-43 found in patient brain. Thus, it has been demonstrated that this insolubilized protein can be utilized as a research tool. A mouse, in which this TDP-43 ΔNLS&FL-A accumulates in the brain thereof, can contribute to elucidation of the pathogenic mechanism of neurodegenerative diseases associated with accumulation of TDP-43 and the development of novel therapeutic drugs for ALS and FTLD.

### REFERENCES

Arai et al, Biochem Biophys Res Commun. 2006 Dec. 22; 351 (3): 602-11.
Buratti and Baralle, J Biol Chem. 2001 Sep. 28; 276 (39): 36337-43.
Neumann et al, Science. 2006 Oct. 6; 314 (5796): 130-3.
Nonaka et al, Cell Rep. 2013 Jul. 11; 4 (1): 124-34.
Watanabe et al, Acta Neuropathol Commun. 2020 Oct. 28; 8 (1): 176.
Wils H et al, Proc Natl Acad Sci USA. 2010 Feb. 23; 107 (8): 3858-63.

### 2. Mutant TDP-43 Protein

The mutant TDP-43 protein of the present invention (simply referred to as “mutant TDP-43” or “mutant,” at times) is a mutant TDP-43 protein, having a deletion of a nuclear localization signal sequence in the amino acid sequence (SEQ ID No: 2, for example, in the case of human) of a wild-type TDP-43 protein, and also having any one of the following mutations (a1) to (c2) or a combination of these mutations:
(a1) a mutation, in which the 147th and 149th phenylalanines are substituted with leucines,
(a2) a mutation, in which the 147th and 149th phenylalanines are substituted with leucines, and one or several amino acids other than the 147th and 149th amino acids are deleted, substituted, or added,
(b1) a mutation, in which the 194th phenylalanine is substituted with leucine,
(b2) a mutation, in which the 194th phenylalanine is substituted with leucine, and one or several amino acids other than the 194th amino acid are deleted, substituted, or added,
(c1) a mutation, in which the 229th and 231st phenylalanines are substituted with leucines, and
(c2) a mutation, in which the 229th and 231st phenylalanines are substituted with leucines, and one or several amino acids other than the 229th and 231st amino acids are deleted, substituted, or added.

The present mutant TDP-43 protein is phosphorylated and has aggregation activity in cells.

In this context, the “nuclear localization signal sequence (NLS)” means an amino acid sequence that serves as a marker for transporting proteins to the cell nucleus, and is also referred to as a “nuclear localizing signal” or “nuclear localization sequence.” In the present description, the nuclear localization signal sequence is sometimes referred to simply as “NLS.” In the amino acid sequence (SEQ ID No: 2 in the case of human) of wild-type TDP-43, NLS is an amino acid sequence ranging from 78th to 84th amino acids, and the mutant TDP-43 protein of the present The mutant TDP-43 protein of the present invention lacks this NLS.

In the present invention, the mutant having the above-described mutation (a1) (F147L and F149L) is referred to as “FL-A.”

Herein, the alphabets before and after the numbers of mutation sites indicate phenylalanine (F) before mutation and leucine (L) after mutation, respectively, and the numbers indicate the existing positions of the mutations.

The mutant having the above-described mutation (b1) (F194L) is referred to as “FL-B.”

The mutant having the above-described mutation (c1) (F229L and F231L) is referred to as “FL-C.”

A mutant having a combination of the mutations (a1) and (b1) is referred to as “FL-AB,” a mutant having a combination of the mutations (a1) and (c1) is referred to as “FL-AC,” a mutant having a combination of the mutations (b1) and (c1) is referred to as “FL-BC,” and a mutant having a combination of the mutations (a1), (b1) and (c1) is referred to as “FL-ABC.”

Moreover, the present invention includes: mutant TDP-43, which has (a2) a mutation in which one or several amino acids other than the 147th and 149th amino acids are deleted, substituted or added, in addition to the above-described mutation (a1); mutant TDP-43, which has (b2) a mutation in which one or several amino acids other than the 194th amino acid are deleted, substituted or added, in addition to the above-described mutation (b1); and mutant TDP-43, which has (c2) a mutation in which one or several amino acids other than the 229th and 231st amino acids are deleted, substituted or added, in addition to the above-described mutation (c1).

Furthermore, the present invention also includes: mutant TDP-43 having each of the above-described mutations (a1) to (c2) alone, and mutant TDP-43 having a combination of these mutations.

However, these mutant TDP-43 have aggregation activity in cells.

Herein, the term “several” indicating the number of amino acid mutations means, for example, 2, 3, 4, 5, 6, 7, 8, or 9 amino acid mutations.

In the present invention, human TDP-43 (SEQ ID No: 2) is exemplified above as an example of TDP-43 to be mutated, but it is also possible to add mutations to TDP-43 derived from other mammals.

Mutation sites corresponding to human TDP-43 and those derived from other mammals are shown in the following Table 1.

TABLE 1

| | Wild-type | Wild-type | | | |
|---|---|---|---|---|---|
| | TDP-43 | TDP-43 | | | |
| | (nucleotide | (amino acid | | | |
| Animal species | sequence) | sequence) | Mutation site | | |
| Human | SEQ ID No: 1 | SEQ ID No: 2 | F147, F149 | F194 | F229, F231 |
| Mouse | SEQ ID No: 3 | SEQ ID No: 4 | F147, F149 | F194 | F229, F231 |
| Rat | SEQ ID No: 5 | SEQ ID No: 6 | F147, F149 | F194 | F229, F231 |
| Marmoset | SEQ ID No: 7 | SEQ ID No: 8 | F147, F149 | F194 | F229, F231 |

Herein, the alphabet F in the mutation site represents phenylalanine, and the number indicates the existing position of the phenylalanine.

### 3. Nucleic Acid Encoding Mutant TDP-43 Protein

### (1) Acquisition of Nucleic Acid Encoding Mutant TDP-43 Protein

The mutant TDP-43 used in the present invention can be obtained by obtaining the gene or amino acid sequence information of wild-type TDP-43 from the accession number, and then performing a known genetic engineering method or site-directed mutagenesis based on the obtained information (Sambrook J. et al., Molecular Cloning, A Laboratory Manual (4th edition), Cold Spring Harbor Laboratory Press (2012)). The wild-type gene can be chemically synthesized to have the nucleotide sequence as set forth in SEQ ID No: 1, 3, 5 or 7, or a commercially available gene can also be used as such a wild-type gene.

The nucleic acid (DNA) encoding the mutant can be obtained, for example, by a known method utilizing site-directed mutagenesis. As a mutagenesis kit for inducing site-specific mutation, for example, QuikChange Site-Directed Mutagenesis Kit (Stratagene), KOD-Plus-Mutagenesis Kit (Toyobo Co., Ltd.), GenEdit Site-Directed DNA Mutagenesis Kit (Funakoshi), TaKaRa Site-Directed Mutagenesis System (Mutan-K, Mutan-Super Express Km, etc.: Takara Bio, Inc. (Takara Bio, Inc.), etc. can be used.

Otherwise, the nucleic acid encoding the mutant TDP-43 can also be produced by conventional chemical or biochemical synthesis methods. For example, a nucleic acid synthesis method, using a DNA synthesizer that has been commonly used as a genetic engineering method, can be used, or after isolating or synthesizing a nucleotide sequence as a template, a PCR method, or a gene amplification method using a cloning vector, can be used. Thereafter, the nucleic acid obtained as described above is cleaved by restriction enzymes, etc. The thus cleaved DNA fragment of the concerned gene is inserted into an appropriate expression vector, so that an expression vector containing the gene encoding the protein can be obtained.

### Construction of Expression Vector and Transformation

In the present invention, the mutant TDP-43 protein can be obtained by introducing a nucleic acid encoding the mutant TDP-43 protein into a vector, as described below, to construct an expression vector (recombinant vector), then introducing the vector into a host, and then culturing the host.

The vector, into which DNA encoding the mutant TDP-43 of the present invention is to be inserted, is not particularly limited, as long as it can replicate in a hos. Examples of the vector may include plasmid DNA, phage DNA, and viruses. Examples of the plasmid DNA may include plasmids derived from E. coli, plasmids derived from Bacillus subtilis, and plasmids derived from yeasts. An example of the phage DNA may be λ phage. Examples of the viruses that can be used herein may include adenoviruses (e.g. adeno-associated virus) and retroviruses. By using such viral vectors, mutants can be introduced into mouse brains or cells.

In the present invention, in addition to promoters and DNA, cis-elements such as enhancers, and splicing signals, poly A addition signals, ribosome-binding sequences (SD sequences), selection marker genes, reporter genes, etc., can be ligated to the expression vector, as desired.

### (3) Collection and Purification of Mutant TDP-43 or its Aggregates

The above-described expression vector is introduced into a host to produce a transformant, the transformed host is cultured or bred, and a mutant TDP-43 protein of interest is collected from the culture product or from the brain of a non-human mammal (e.g. a mouse). The term “culture product” is used to mean any of (i) a culture supernatant, and (ii) cultured cells or a cultured cell mass, or crushed products thereof.

### 4. Cell Models and Animal Models for Mutant TDP-43 Accumulation

As described above, when a vector expressing the mutant TDP-43 (adeno-associated virus, AAV) is inoculated into mouse brain, phosphorylated TDP-43 antibody-positive intracellular aggregates are observed in the brain, and also, insolubilization of phosphorylated TDP-43 is biochemically confirmed. When the insolubilized TDP-43 aggregates prepared from the mouse brain are introduced into ΔNLS-TDP-43-expressing cultured cells, the insolubilized TDP-43 aggregates are function as seeds for triggering intracellular accumulation of TDP-43, and accumulation of TDP-43 is enhanced in cultured cells. The mouse accumulating phosphorylated TDP-43 in the brain thereof, which has been constructed in the present invention, can be used as a mouse model of neurodegenerative disease for elucidation of the mechanism of intracellular accumulation of TDP-43 and the development of therapeutic drugs such as compounds for suppressing TDP-43 accumulation. In addition, since the insolubilized TDP-43 prepared from this mouse brain functions as a seed to induce the accumulation of the TDP-43, the cells, into which mutant TDP-43 aggregates accumulated in the mouse brain have been introduced, can be utilized as a cell model for neurodegenerative disease.

In the present invention, the animal is not limited to a mouse, and other animals can also be applied. The targets of animal models may include rats, rabbits, dogs, horses, sheep, marmosets, and monkeys (macaques).

### 5. Method of Screening for Therapeutic Drug for Neurodegenerative Disease

The screening method of the present invention is characterized in that it comprises allowing a candidate substance (test substance) to come into contact with the animal or cell of the following (a) to (d):
(a) a mutant human TDP-43-expressing non-human mammal,
(b) a mutant non-human TDP-43-expressing non-human mammal,
(c) a cell, into which a mutant TDP-43 aggregate derived from the brain of the above-described animal (a) or (b) has been introduced, or
(d) a non-human mammal.

Thereby, it becomes possible to screen for a substance that suppresses the intracellular accumulation of mutant TDP-43 aggregates, and it also becomes possible to screen for a therapeutic drug for neurodegenerative diseases.

Examples of the target diseases of the medicine as a target of screening may include amyotrophic lateral sclerosis, frontotemporal lobar degeneration, and other diseases attended with accumulation of TDP-43 (e.g., Alzheimer's disease, Lewy body dementia, etc. attended with TDP-43 accumulation).

The term “contact” means that a cell or a non-human mammal, into which mutant TDP-43 protein or an aggregate thereof has been introduced, and a candidate substance (test substance), are allowed to exist in the same environment, reaction system, or culture system. Examples thereof may include: adding a candidate substance into a cell culture vessel; mixing a cell with a candidate substance; culturing a cell in the presence of a candidate substance; and administering a candidate substance to a non-human mammal.

Examples of the candidate substance may include peptides, proteins (including antibodies), non-peptide compounds, synthetic compounds (high-molecular-weight or low-molecular-weight compounds), fermentation products, cell extracts, cell culture supernatants, plant extracts, tissue extracts of mammals (e.g., mice, rats, pigs, bovines, sheep, monkeys, humans, etc.), and blood plasma. These compounds may be either novel compounds or known compounds. These candidate substances may form salts, and examples of the salts of the candidate substances may include salts with physiologically acceptable acids (e.g., inorganic acids, organic acids, etc.) or with bases (e.g., metallic acids, etc.).

If the results show that alleviation or elimination of cell death can be confirmed when a certain candidate substance is administered, the candidate substance used therein can be selected as a therapeutic drug for neurodegenerative disease.

### 5. Kit Used in Method of Screening for Therapeutic Drug for Neurodegenerative Disease

The cell model of the present invention can be provided in the form of a kit for screening for a therapeutic drug for neurodegenerative disease. The kit of the present invention includes the above-mentioned cells, but can also include labeling substances, reagents for cell death detection (e.g., LDH, etc.), and the like. The term “labeling substance” means enzymes, radioisotopes, fluorescent compounds, chemiluminescent compounds, etc. The kit of the present invention can include, in addition to the above-described constituent elements, other reagents for carrying out the method of the present invention, such as enzyme substrates (chromogenic substrates, etc.), enzyme substrate lysates, enzyme reaction stopper solutions, etc. when the labeling is an enzyme labeling. Furthermore, the kit of the present invention can also include diluents for test compounds, various types of buffers, sterile water, various types of cell culture vessels, various types of reaction vessels (e.g. Eppendorf tubes, etc.), detergents, experimental operating manuals (instructions), etc.

### EXAMPLES

Hereinafter, the present invention will be more specifically described in the following examples. However, these examples are not intended to limit the scope of the present invention.

### Example 1

### Cultured Cells

The human neuroblastoma cell line SH-SY5Y (American Type Culture Collection, Cat. #CRL-2266) purchased from the American Type Culture Collection was used. Using, as a culture medium, DMEM (Dulbecco's modified eagle's medium nutrient mixture)/F-12HAM (Sigma-Aldrich), which was supplemented with 10% (v/v) fetal bovine serum, a non-essential amino acid solution (MEM Non-Essential Amino Acids Solution (100×), ThermoFisher, Cat. #11140050) and a penicillin-streptomycin-glutamine solution (Penicillin-Streptomycin-Glutamine (100×), ThermoFisher, Cat. #10378016), the cells were cultured at 37° C. in a 5% CO₂ incubator (Thermo SCIENTIFIC). For the culture, a collagen-coated 6-cm petri dish (BD Biocoat) and a 6-well plates (BD Biocoat) were used. The Cell passaging was performed according to the following procedures, in a state in which the cells were 100% confluent. After removing the medium from the 6-cm petri dish, the cells were washed with 1.5 mL of a normal saline, and the normal saline was then removed. Thereafter, 1 mL of 0.25% trypsin was added to the cells, and the obtained mixture was then kept warm at 37° C. for 5 minutes. Thereafter, 2 mL of a fresh medium was further added, and after the trypsin reaction was terminated, the cells were thoroughly suspended and were seeded in 6-cm petri dish added with 3 mL of medium. In general, when 4 to 6×10⁵ cells are added to a 6-cm petri dish containing 3 mL of medium, the cells become almost 100% confluent (2 to 3×10⁶ cells/mL) in about 3 days.

For preparation of AAV, HEK293T cells were used. Using, as a culture medium, DMEM (Dulbecco's modified eagle's medium nutrient mixture (high glucose), Sigma-Aldrich, #D5796-500ML), supplemented with 10% (v/v) fetal bovine serum, a non-essential amino acid solution (MEM Non-Essential Amino Acids Solution (100×), ThermoFisher, Cat. #11140050), a penicillin-streptomycin-glutamine solution (Penicillin-Streptomycin-Glutamine (100×), ThermoFisher, Cat. #10378016) and sodium pyruvate (GIBCO, #11360), the cells were cultured at 37° C. in a 5% CO₂ incubator (Thermo SCIENTIFIC).

### Preparation of Mutant TDP-43

Plasmids encoding mutant TDP-43 were produced using the QuikChange Site-Directed Mutagenesis Kit (Strategene). Upon production of a mutant in which the nuclear localization signal (NLS) was removed (a ΔNLS mutant) and a mutant in which Phe involved in RNA binding was substituted with Leu (an FL mutant), pcDNA3-TDP-43 (a cultured cell expression vector) encoding wild-type TDP-43 was used as a template, and PCR was performed using the following primers. For production of a ΔNLS&FL-A mutant, PCR was performed using pcDNA3-TDP ΔNLS as a template with the following primers.

ΔNLS-FW
(SEQ ID No: 9)
GTATGTTGTCAACTATATGGATGAGACAGATGC
ΔNLS-RV
(SEQ ID No: 10)
GCATCTGTCTCATCCATATAGTTGACAACATAC
FL-A(F147 & 149L)-FW
(SEQ ID No: 11)
AGACTGGTCATTCAAAGGGGTTGGGCTTGGTTCGTTTTACGGAATATGA
FL-A-RV
(SEQ ID No: 12)
TCATATTCCGTAAAACGAACCAAGCCCAACCCCTTTGAATGACCAGTCT
FL-B(F194L)-FW
(SEQ ID No: 13)
CTTTGAGAAGCAGAAAAGTGTTGGTGGGGCGCTGTACAGAGGA
FL-B-RV
(SEQ ID No: 14)
TCCTCTGTACAGCGCCCCACCAACACTTTTCTGCTTCTCAAAG
FL-C(F229 & 231L)-FW
(SEQ ID No: 15)
TCCCCAAGCCATTCAGGGCCTTGGCCTTGGTTACATTTGCAGATGATCA
FL-C-RV
(SEQ ID No: 16)
TGATCATCTGCAAATGTAACCAAGGCCAAGGCCCTGAATGGCTTGGGGA

### Construction of AAV Expression Vector

The vector for AAV expression was prepared by performing PCR using wild-type or mutant pcDNA3-TDP-43 as a template with the following primers, and the wild-type or mutant TDP-43 sequence was then introduced into the EGFP portion of AAV-hsyn-EGFP (Addgene, #114213) having a synapsin promoter.

hSyn-TDP-FW
(SEQ ID No: 17)
GGATCCGCCGCCACCATGTCTGAATATATTCGGGTA
hSyn-TDP-RV
(SEQ ID NO: 18)
CGCGGCCGCACTAGTCTACATTCCCCAGCCAGA

PCR was performed using Prime STAR Max Premix (Takara, R045A). PCR products were electrophoresed on an agarose gel, and bands of interest were then cut out. Thereafter, DNA was extracted using NucleoSpin Gel and PCR Clean-up (Takara, U0609B). The extracted DNA was cyclized using In-Fusion HD Cloning kit (Takara, #639648), and was used as an expression vector (the vector expressing wild-type TDP-43 was AAV-hsyn-TDP wild-type, whereas a mutant without nucleic acid-binding ability was AAV-hsyn-TDP FL-A).

### Preparation of AAV

HEK293T cells were seeded in an amount of 2.0×10⁶ cells each on ten 10-cm petri dishes. To the cells, a mixture consisting of an AAV vector (AAV-hsyn-TDP wild-type or FL-A), an AAV-PHP.eB vector (a plasmid for determining AAV serotype), an AAV helper plasmid pAdDeltaF6 (Addgene, #112867) and PEI (Polysciences, Inc.) was added. Specifically, per 10-cm petri dish, 2.5 μg of the AAV vector, 5 μg of pΔF6, 2.5 μg of the AAV-PHP.eB, and 50 μL of the 1 mg/mL PEI were added to and mixed with 1 mL of Opti-Mem (gibco Opti-MEM #11058-021), and the thus obtained mixture was left at rest at room temperature for 20 minutes. Thereafter, the total amount of the mixed solution was added into the culture medium in the petri dish for transfection. After 2 days, the total amount of the medium was exchanged with a serum-free DMEM medium. After 4 days, the medium was collected and were centrifuged at 4500 rpm for 5 minutes, and the cells were removed. The medium was further filtrated through a 0.45-μm filter (Thermo Nalgene Rapid-Flow Filters, #165-0045) to remove excess cell debris and the like, and was then transferred into a centrifuge tube (Millipore Amicon Ultra, #UFC910024), and it was then centrifuged at 5000 g until about 800 μL remained on the membrane. Thereafter, 10 mL of PBS was added onto the membrane, and the mixture was centrifuged at 5000 g until about 800 μL remained on the membrane. After repeating this operation three times, the virus solution on the membrane was collected and was freeze-preserved at −80° C.

### Measurement of AAV Titer

A WPRE sequence specific to AAV was amplified by real-time PCR using the following primers.

FW
(SEQ ID No: 19)
TGGCGTGGTGTGCACTGT
RV
(SEQ ID No: 20)
AGGGACGTAGCAGAAGGACG

As a sample for PCR, 2 μL of an AAV sample was mixed with a PCR alkaline treatment buffer (25 mM NaOH, 0.2 mM EDTA), and the mixture was heat-treated at 100° C. for 10 minutes using a thermal cycler (BioRAD). Then, 50 μL of a PCR neutralization buffer (1 M Tris-HCl, pH 5.0) was added to the reaction mixture, and was then blended by pipetting. Thereafter, 2 μL of the obtained mixture, 10 μL of SYBR Green (Thermo scientific, #4367659), 2 μL of primers, and 6 μL of DW were mixed with one another, and the thus obtained mixture was analyzed by real-time PCR. The ABI 7500 Fast (ThermoFisher, #4406984) was used as a device herein, and the first cycle was carried out at 25° C. for 10 minutes and the following 40 cycles were carried out by repeating at 95° C. for 15 seconds, at 60° C. for 30 seconds, and at 72° C. for 30 seconds.

The plasmid concentration and the copy number were obtained from the following expression:

[Plasmid concentration ng/μL (1.2×10¹⁵)]/(base number bp*607.4)+157.9].

AAV-hsyn-EGFP (Addgene #114213), whose plasmid concentration and base number had been known, was serially diluted, and a calibration curve of copy number and reaction time was prepared. The copy number was calculated from the reaction time of the purified virus solution.

AAC Infection into Mice

Wild-type male mice (C57BL/6J) were purchased from Japan SLC, Inc., and were bred at the animal breeding facility of The Tokyo Metropolitan Institute of Medical Science.

The samples inoculated into mouse brains were as follows
1. AAV prepared from the AAV-hsyn-EGFP vector, 1.0×10⁸ vg or 1.0×10⁹ vg (negative control)
2. AAV hsyn-TDP43ANLS&FL-A , 1.0×10⁸ vg or 1.0×10⁹ vg

The mice were placed in an anesthesia chamber, and the chamber was filled with an anesthetic at 3% (20% isoflurane, Japanese Pharmacopoeia, Pfizer) and was left at rest for several minutes. Thereafter, the anesthetized mice were immobilized with auxiliary ear bars and were further anesthetized with 2% isoflurane by inhalation. The above-described 4 types of AAVs were inoculated as samples into the bilateral striatum of the mice as follows.

First, the scalp of each mouse was incised, and the location of the bregma was confirmed. Then, holes were drilled into the skull at points that were 2 mm to the left and right and 0.5 mm vertically from the location of the bregma. A syringe (HAMILTON, Cat. #80301) containing the sample was inserted into a depth of 3 mm such that the needle thereof was prevented from bending, and 5 μL of the sample was inoculated. In order to reduce the leakage of the sample, the syringe was left at rest in the place for 1 minute, the needle was then removed, and thereafter, the sample was also inoculated into the hole on the opposite side. Thereafter, the incised scalp was then sutured, and finally, for identification purposes, the ears were punched and the mice was returned to the cage.

### Excision of Mouse Brain

One month after the inoculation of mouse brains with the sample, the brains were removed. The mice were anesthetized by injecting 0.5 mL of three types of mixed anesthetics into the abdominal cavity. The three types of mixed anesthetics were prepared by mixing 750 μL of medetomidine hydrochloride (Domitor, Nippon Zenyaku Kogyo Co., Ltd.), 800 μL of mitazolam (Sandoz Pharma K.K.), and 1 mL butorphanol tartrate (Betrufal, Meiji Seika Pharma Co. Ltd.) into 7.45 mL of a normal saline (Japanese Pharmacopoeia normal saline solution, OTSUKA NORMAL SALINE, Otsuka Pharmaceutical Factory). After the anesthetic treatment, hemorrhage was performed from the whole body with a normal saline (Terumo Normal Saline, B type, Terumo), and the brain was then removed.

### Preservation of Mouse Brain

The mouse brain was immediately placed on a petri dish and was divided into right and left halves along the median line with a feather steel blade (black blade). The right brain was frozen with dry ice and was preserved at −80° C. The left brain was fixed at 4° C. in a 10% neutral buffered formalin solution (FUJIFILM Wako Pure Chemical Corporation, Cat. #062-01661). Tissues were fixed as quickly as possible (in 2 to 3 days) because of the risk that the tissues were not only disintegrated but an antigen of interest was leaked out over time.

### Paraffin Block Embedding

The fixed mouse brains were placed in a paraffin block embedding cassette (Sakura Tissue-Tek #4143), and were immersed in paraffin using an automated paraffin embedding device (SAKURA SEIKI Co., Ltd., Tissue-Tek VIP5 Junior) according to the protocol in Channel 1. Thereafter, the mouse brain and liquid paraffin were poured into a metal mold for paraffin-embedding, and were then solidified at 0° C. to prepare paraffin blocks.

### Vibratome Section Preparation and Immunohistochemical Analysis

Using a thin sectioning device (YAMATO KOHKI INDUSTRIAL CO., LTD., Littratome, #REM-710) in which a microtome blade (Feather, Microtome Blades, #S35 TYPE) was attached to a tissue blade holder (YAMATO KOHKI INDUSTRIAL CO., LTD., Hard Tissue Blade Holder, #BH-220), the mouse brains embedded in the paraffin blocks were sliced at a thickness of 8 μm to prepare sections. These sections were attached to anti-exfoliation coated glass slides (MATSUNAMI). Subsequently, these sections were permeated with xylene for 20 minutes, and then with ethanol for 20 minutes, so as to dissolve the paraffin.

The sections were washed in running water for 5 minutes to rinse off the remaining organic solvent, and the resulting sections were then subjected to an autoclave treatment in a 0.01 M sodium citrate buffer at 121° C. for 20 minutes. Thereafter, the sections were immersed in PBS containing 3% H₂O₂ and were washed three times with PBS. Blocking was performed in 10% bovine serum (CS: Bovine Serum, ThermoFisher, Cat. #16170-078) containing 0.03% Triton X-100 for 20 minutes, and the resultant was then allowed to react overnight with a 6,000-fold diluted human specific TDP-43 antibody (Proteintech, Cat. #60019-2-Ig), or with a 1,000-fold diluted phosphorylated TDP-43 antibody (Cosmo Bio Co., Ltd., Cat. #TIP-PTD-P07).

The next day, the sections were washed three times with PBS, and were then allowed to react together with a secondary antibody (1:1,000 dilution; Biotin-Goat anti mouse IgG, Vector, Cat. BA-9200-1.5, or Biotin-Goat anti-rabbit IgG, Vector, Cat. #BA-1000-1.5) at room temperature for 2 hours. Thereafter, the sections were washed three times with PBS, and were then allowed to react with an avidin-biotin-peroxidase complex, using the ABC Kit (peroxidase-labeled avidin-biotin complex (ABC Standard Kit, Vector, Cat. #PK-4000)) at room temperature for 30 minutes. Thereafter, the reaction mixture was washed three times with PBS, and the resultant was colored with a prepared chromogenic solution (a Tris normal saline containing 0.1% 3,3′-Diaminobenzidine (Sigma-Aldrich, Cat. #D8001-5G) and 0.05% H₂O₂ (hydrogen peroxide, Sigma-Aldrich Japan, Cat. #13-1910-5)) for 20 minutes.

The coloration reaction was terminated by washing the section-attached glass slides with tap water. After drying these sections, they were treated with Mayer's Hematoxylin (MUTO PURE CHEMICALS CO., LTD., Cat. #30002) for 1 minute to perform nuclear staining, and were then washed with running water for 5 minutes. The specimens were completely dried, were immersed in xylene for 10 minutes for dehydration, and were then encapsulated using Antifade Mounting Medium for fluorescence (VECTASHIELD, Cat. #H-1000-10). The prepared specimens were preserved at room temperature. An all-in-one microscope (KEYENCE: BZ-X710) was used for observation.

### Detection of Phosphorylated TDP-43 by Immunoblotting

Frozen mouse brain was homogenized in an A68 buffer (10 mM Tris-HCl, pH 7.5/1 mM EGTA/10% sucrose/0.8 M NaCl) in an 18-fold volume per 0.25 g of the brain. To the suspension, a 20% sarcosyl solution (N-lauroyl sarcosine sodium salt, Sigma-Aldrich, Cat. #L5125-500G) was added to result in a final concentration of 1%, and the obtained mixture was stirred and was then kept warm at 37° C. for 30 minutes. Thereafter, this suspension was centrifuged (TOMY MX-307, TOMY SEIKO CO., LTD.) at 20,400 g at room temperature for 10 minutes, and the supernatant was then collected. The supernatant was further centrifuged at 113,000 g for 20 minutes (himac CS100GXL, Eppendorf Himac Technologies Co., Ltd.). The supernatant was collected as a sarcosyl supernatant fraction (Sar-sup). To the precipitate, 500 μL of PBS was added, and the obtained mixture was then centrifuged again at 113,000 g for 20 minutes (himac CS100GXL, Eppendorf Himac Technologies Co., Ltd.). Thereafter, the supernatant was discarded, and the precipitate was collected as a sarcosyl-insoluble fraction (Sar-ppt).

The sarcosyl supernatant fraction was subjected to BCA assay (BCA Protein Assay Kit, ThermoFisher, Cat. #23225) to quantify the amount of protein in the supernatant fraction. To 100 μL of the remaining sample, 100 μL of a 2-fold-concentration SDS sample buffer (2×SB) containing 5% 2-mercaptoethanol was added. To the sarcosyl-insoluble fraction, 50 μL of 2×SB containing 5% 2-mercaptoethanol was added, and the obtained mixture was then subjected to an ultrasonic treatment (TAITEC VP-050 ultrasonic treatment device, TAITEC CORPORATION). The reaction mixture was subjected to a heat treatment at 100° C. for 5 minutes, followed by recovery.

The obtained samples (Sar-sup and Sar-ppt) were electrophoresed on a 13.5% polyacrylamide gel, and were each transferred onto a PVDF membrane (Millipore) under conditions of 200 mA and 1 hour. The PVDF membranes were blocked with a normal saline containing 3% gelatin (FUJIFILM Wako Pure Chemical Corporation, Cat. #077-03155) at room temperature for 10 minutes, and were then allowed to react with a primary antibody (1:6,000 dilution; a human-specific TDP-43 antibody (Proteintech, Cat. #60019-2-Ig) or with a phosphorylated TDP-43 antibody (1:1,000 dilution; Cosmo Bio Co., Ltd., Cat. #TIP-PTD-P07), which had been diluted with a normal saline containing 10% bovine serum (CS: Bovine Serum, ThermoFisher Cat. #16170-078) and 0.1% NaN₃ (10% CS/PBS), at room temperature overnight.

Thereafter, the PVDF membranes were washed with several mL of PBS, and were then allowed to react with a secondary antibody diluted with 10% CS/normal saline (1:1,000 dilution; Biotin-Goat anti mouse IgG, Vector, Cat. #BA-9200-1.5 or Biotin-Goat anti rabbit IgG, Vector, Cat. #BA-1000-1.5) at room temperature for 1 hour, and were then washed with PBS. The PDVF membranes were allowed to react with a peroxidase-labeled avidin-biotin complex (ABC Standard Kit, Vector, Cat. #PK-4000) for 30 minutes, and were then washed with PBS. After that, the PDVF membranes were treated with PBS containing 0.1% 3,3′-Diaminobenzidine (Sigma-Aldrich, Cat. #D8001-5G), 0.2 mg/mL Nickel (II) chloride hexahydrate (FUJIFILM Wako Pure Chemical Corporation, Cat. #141-01045) and 0.05% H₂O₂ (hydrogen peroxide water, Sigma-Aldrich Japan, Cat. #13-1910-5), and the protein bands on the membranes were colored for 10 minutes. The coloration reaction was terminated by washing the PVDF membranes with DW.

### Studies of Seeding Activity of Sarcosyl-Insoluble Fraction:

### Preparation of Seed Fraction (Sarcosyl-Insoluble Fraction)

A frozen mouse brain was homogenized in an A68 buffer (10 mM Tris-HCl, pH 7.5/1 mM EGTA/10% sucrose/0.8 M NaCl) in an 18-fold volume per 0.25 g of the mouse brain. To the suspension, a 20% sarcosyl solution (N-lauroyl sarcosine sodium salt, Sigma-Aldrich, Cat. #L5125-500G) was added to result in a final concentration of 1%, and the obtained mixture was stirred and was then kept warm at 37° C. for 30 minutes. Thereafter, this suspension was centrifuged (TOMY MX-307, TOMY SEIKO CO., LTD.) at 20,400 g at room temperature for 10 minutes, and the supernatant was then collected. The supernatant was further centrifuged at 113,000 g for 20 minutes (himac CS100GXL, Eppendorf Himac Technologies Co., Ltd.).

The supernatant was collected as a sarcosyl supernatant fraction (Sar-sup). To the precipitate, 500 μL of PBS was added, and the obtained mixture was then centrifuged again at 113,000 g for 20 minutes (himac CS100GXL, Eppendorf Himac Technologies Co., Ltd.). Thereafter, the supernatant was discarded, and the precipitate was collected as a sarcosyl-insoluble fraction (Sar-ppt). To the obtained sarcosyl-insoluble fraction, 50 μL of a normal saline was added, and the obtained mixture was then subjected to an ultrasonic treatment using TAITEC VP-050 ultrasonic treatment device. The obtained reaction mixture was used as a mouse brain-derived seed fraction. On the other hand, a sarcosyl-insoluble fraction was obtained by the same method as that described above, using, as a positive control, a frozen brain sample (0.5 g) from an ALS patient brain. To the thus obtained fraction, 50 μL of a normal saline was added, and the obtained mixture was then subjected to an ultrasonic treatment using TAITEC VP-050 ultrasonic treatment device. The obtained reaction mixture was used as a human brain-derived seed fraction.

### Addition of Seed Fraction to Cells

Regarding the seeding activity of a TDP-43ΔNLS&FL-A expressing mouse brain sarcosyl-insoluble fraction, using cultured cells, the following procedures were used.

That is, 8×10⁵ SH-SY5Y cells were seeded per well on a 6-well plate, and were then cultured overnight. The next day, the TDP-43 ΔNLS mutant expression plasmid (pcDNA3-TDP-43 ΔNLS) was introduced into the cells, using X-treamGENE9 (Roche, Cat. #6365809001). Specifically, Opti-MEM (ThermoFisher, Cat. #31985062), the plasmid, and the X-treamGENE9 were gently mixed with one another at a ratio of 100 μL:1 μL:3 μL, and were then left at rest at room temperature for 15 minutes. Thereafter, the mixed solution was dropped into the culture medium in each well. After 3 to 5 hours had been passed, 5 μL of mouse brain-derived seed, or human brain-derived seed used as a positive control, was added to the culture medium in each well. The treated cells were incubated in a CO² incubator, and 2 days later, the cells were collected as follows.

### Detection of Insolubilized TDP-43 by Immunoblotting Method

The culture medium in each well was removed with an aspirator, to which 1 mL of normal saline was added and the cells were peeled from the plate and were collected. The cells were collected by centrifugation at 1,800 g for 5 minutes, and 300 μL of an A68 buffer (10 mM Tris-HCl, pH 7.5/1 mM EGTA/10% sucrose/0.8 M NaCl) containing 1% sarcosyl (N-lauroyl sarcosine sodium salt, Sigma-Aldrich, Cat. #L5125-500G) was added to the collected cells. Then, the obtained mixture was treated using TAITEC VP-050 ultrasonic treatment device (PWM 17% intensity) for 40 to 60 seconds, so as to crush the cells.

Thereafter, 300 mL of an A68 buffer containing 1% sarcosyl was further added to the resulting cells, and the cells were then centrifuged (himac CS100GXL, Eppendorf Himac Technologies Co., Ltd.) at 113,000 g for 20 minutes. The obtained supernatant (100 μL) was collected, and 100 μL of a 2-fold-concentration SDS sample buffer (2×SB) containing 5% 2-mercaptoethanol was added thereto. The obtained solution was heat-treated at 100° C. for 5 minutes, and a sarcosyl supernatant fraction (Sar-sup) was collected. Further, using 15 μL of the supernatant, BCA assay (BCA Protein Assay Kit, ThermoFisher, Cat. #23225) was performed, so that the amount of protein in the supernatant fraction was quantified.

On the other hand, the precipitate fraction was subjected to an ultrasonic treatment after addition of 50 μL of 2×SB containing 5% 2-mercaptoethanol, and was then heat-treated at 100° C. for 5 minutes, so that the resultant was collected as a sarcosyl-insoluble fraction (Sar-ppt). The obtained samples (Sar-sup and Sar-ppt) were electrophoresed on a 13.5% polyacrylamide gel, and were each transferred onto a PVDF membrane (Millipore) under conditions of 200 mA and 1 hour. The PVDF membranes were blocked with a normal saline containing 3% gelatin (FUJIFILM Wako Pure Chemical Corporation, Cat. #077-03155) at room temperature for 10 minutes, and were then allowed to react with a primary antibody (1:6,000 dilution; a human-specific TDP-43 antibody (Proteintech, Cat. #60019-2-Ig) or with a phosphorylated TDP-43 antibody (1:1,000 dilution; Cosmo Bio Co., Ltd., Cat. #TIP-PTD-P07), which had been diluted with a normal saline containing 10% bovine serum (CS: Bovine Serum, ThermoFisher Cat. #16170-078) and 0.1% NaN₃ (10% CS/normal saline), at room temperature overnight.

Thereafter, the PVDF membranes were washed with several mL of PBS, and were then allowed to react with a secondary antibody diluted with 10% CS/normal saline (1:1,000 dilution; Biotin-Goat anti mouse IgG, Vector, Cat. #BA-9200-1.5 or Biotin-Goat anti rabbit IgG, Vector, Cat. #BA-1000-1.5) at room temperature for 1 hour, and were then washed with PBS. The PDVF membranes were allowed to react with a peroxidase-labeled avidin-biotin complex (ABC Standard Kit, Vector, Cat. #PK-4000) for 30 minutes, and were then washed with PBS. After that, the PDVF membranes were treated with PBS containing 0.1% 3,3′-Diaminobenzidine (Sigma-Aldrich, Cat. #D8001-5G), 0.2 mg/mL Nickel (II) chloride hexahydrate (FUJIFILM Wako Pure Chemical Corporation, Cat. #141-01045) and 0.05% H₂O₂ (hydrogen peroxide water, Sigma-Aldrich Japan, Cat. #13-1910-5), and the protein bands on the membranes were colored for 10 minutes. The coloration reaction was terminated by washing the PVDF membranes with DW.

### Results:

The relationship between the RNA-binding ability and the aggregation properties of TDP-43 was examined. It has been reported that the nucleic acid-binding ability of TDP-43 disappears when several Phe residues existing in the RNA-binding domain of TDP-43 are substituted with Leu residues (FIG. 1). Thus, when mutants (FL mutants, FIG. 1) in which these Phe residues were substituted with Leu residues were prepared, and were transiently allowed to expressed in cultured cells, phosphorylated TDP-43 antibody-positive intracellular aggregates were observed (FIG. 2). Furthermore, according to the biochemical analysis of FL mutant-expressing cell lysates (FIG. 3), in FL-A mutant-expressing cells, a phosphorylated TDP-43 antibody-positive band was observed in the detergent sarkosyl (Sar) insoluble fraction (Sar-ppt), and it was confirmed that this mutant most strongly aggregates in the cells. Moreover, in cells expressing the mutant (ΔNLS&FL-ABC) having nuclear localization signal (NLS) deletion mutation (ΔNLS) as well as FL mutation, significant intracellular accumulation of phosphorylated TDP-43 was observed, compared with FL-A mutant-expressing cells.

From the aforementioned results, it became clear that when FL mutants involved in the nucleic acid binding of TDP-43 were allowed to express in cultured cells, phosphorylated TDP-43 accumulates in the cells. Moreover, considering that FL-A exhibits the strongest aggregation properties among the FL mutants (FIG. 1), and further that the accumulation thereof is significantly increased by introducing a mutation involving deletion of NLS into the cells (FIG. 3), a new mutant, TDP-43 ΔNLS&FL-A, was prepared by introduction of the two above mutations. Thereafter, whether intracellular accumulation of phosphorylated TDP-43 occurs even in mouse brains by allowing the aforementioned new mutant to express in the mouse brains was examined. AAV virus (serotype: AAV-PHP.eB) expressing TDP-43 ΔNLS&FL-A was prepared using HEK293 cells according to an ordinary method. The medium of the plasmid-transfected cells was collected, a virus solution was concentrated by an ultrafiltration method, and its concentration was measured by a real-time PCR method. 1.0×10⁸ vg or 1.0×10⁹ vg of the obtained virus was inoculated into the bilateral striatum of adult mouse brain. One month or three months after the inoculation of the virus, the brain was excised, and then, the right brain was subjected to biochemical analysis (immunoblotting) and the left brain to immunohistochemical analysis (FIG. 4).

One month after the infection with AAV, the mouse brain (right brain) was homogenized in a buffer containing sarcosyl, and was then centrifuged. The obtained supernatant (soluble fraction: Sar-sup) and precipitate (insoluble fraction: Sar-ppt) were collected and were subjected to immunoblot analysis. According to the analysis of the Sar-sup fraction shown in FIG. 5, the expression of TDP-43 ΔNLS&FL-A of interest was observed in virus-infected mice (the red arrow bands in FIG. 5). The expression level was dependent on the amount of the virus inoculated into the mouse brain, and a higher expression was observed in the mouse brain infected with 1.0×10⁹ vg of AAV. On the other hand, the expression was not observed in mice inoculated with a normal saline instead of the virus. Subsequently, in the results of the immunoblotting of the Sar-ppt fraction shown in FIG. 6, a phosphorylated TDP-43-specific antibody-positive band was observed in the sarcosyl-insoluble fraction of mouse brain infected with 1.0×10⁹ vg of AAV (the red arrow). The intensity of the band varied among individuals, but these bands were not detected in mouse brains infected with a normal saline or 1.0×10⁸ vg of AAV. From these results, it became clear that TDP-43 ΔNLS&FL-A expressed in brain by AAV infection is phosphorylated and aggregated in the cells.

From the aforementioned results of the biochemical analysis of AAV-infected mouse brains, it was found that the expression of TDP-43 ΔNLS&FL-A was significantly observed in mice infected with 1.0×10⁹ vg of the virus. Thus, the immunohistochemical analysis of these mouse brains was conducted. One month after infection with 1.0×10⁹ vg of AAV, the mouse brains were excised, and their left brains were fixed in formalin. Thereafter, paraffin blocks were prepared, and ultrathin slices (i.e. 8-μm thick slices) were prepared and stained with human TDP-43-specific antibodies and phosphorylated TDP-43-specific antibodies. As a result, as shown in FIG. 7, the expression of TDP-43 ΔNLS&FL-A was observed in the cerebellum, the cerebral cortex, the hippocampus and the medial posterior parietal cortex by the AAV virus infection. In addition, the sections of the AAV-infected mouse brains were stained using phosphorylated TDP-43-specific antibodies specifically reacting with phosphorylated TDP-43 aggregates appearing in the patient brains. As a result, the phosphorylated TDP-43-specific antibody-positive staining images were observed in the hippocampus and the medial posterior parietal cortex (FIG. 8). From these results, it became clear that phosphorylated TDP-43 ΔNLS&FL-A forms intracellular aggregates in this AAV-infected mouse brain, and that a novel mouse model that faithfully reflects the TDP-43 pathology of the patient brain can be constructed.

TDP-43 accumulated in the brain of ALS or FTLD patients has been reported to have prion-like properties (6). That is, when insolubilized TDP-43 prepared as a sarcosyl-insoluble fraction from the patient brain is introduced into cultured cells expressing TDP-43 NLS, it functions as a seed to induce aggregation of TDP-43 ΔNLS TDP-43 ΔNLS in the cells (FIG. 9; Lane 6). Thus, whether TDP-43 ΔNLS&FL-A aggregated in the AAV-infected mouse brain also has similar prion-like properties, namely, whether it can function as a seed, was examined.

50 μL of a normal saline was added to sarcosyl-insoluble fractions (Lane 4, etc. of FIG. 6) prepared from AAV-infected mouse brains, and the resulting factions were then subjected to an ultrasonic treatment. The thus obtained fractions were used as insolubilized TDP-43 ΔNLS& FL-A. To SH-SY5Y cells that had previously expressed TDP-43 ΔNLS, 5 μL of insolubilized TDP-43 ΔNLS& FL-A was added together with MultiFectam reagent (Promega). After incubation for several days, the cells were collected, were homogenized in a sarcosyl-containing buffer, and were centrifuged to obtain a sarcosyl-insoluble fraction (Sar-ppt). The Sar-ppt was subjected to immunoblot analysis using a phosphorylated TDP-43-specific antibody.

Even if TDP-43 ΔNLS is expressed alone in cultured cells, it does not accumulate in the cells and therefore only few bands are observed in the Sar-ppt fraction (FIG. 9; Lane 3). On the other hand, when insolubilized TDP-43 prepared from ALS patient brains is added as a seed to cells, it functions as a seed and induces accumulation of TDP-43 ΔNLS in the cells (FIG. 9; Lane 6). Similarly, when insolubilized TDP-43 ΔNLS&FL-A prepared from AAV-infected mouse brains was added to cells, phosphorylated TDP-43-specific antibody-positive bands appeared in the Sar-ppt fraction (FIG. 9; Lanes 4 and 5).

Therefore, it became clear that the insolubilized TDP-43 ΔNLS&FL-A observed in the AAV-infected mouse brains has prion-like properties, as with the insolubilized TDP-43 observed in the patient brains.

1. A mutant TDP-43 protein, having a deletion of a nuclear localization signal sequence in the amino acid sequence of a wild-type TDP-43 protein, and also having any one of the following mutations (a1) to (c2) or a combination of these mutations, wherein
the mutant TDP-43 protein has aggregation activity in cells:
(a1) a mutation, in which the 147th and 149th phenylalanines are substituted with leucines,
(a2) a mutation, in which the 147th and 149th phenylalanines are substituted with leucines, and one or several amino acids other than the 147th and 149th amino acids are deleted, substituted, or added,
(b1) a mutation, in which the 194th phenylalanine is substituted with leucine,
(b2) a mutation, in which the 194th phenylalanine is substituted with leucine, and one or several amino acids other than the 194th amino acid are deleted, substituted, or added,
(c1) a mutation, in which the 229th and 231st phenylalanines are substituted with leucines, and
(c2) a mutation, in which the 229th and 231st phenylalanines are substituted with leucines, and one or several amino acids other than the 229th and 231st amino acids are deleted, substituted, or added.

2. The mutant TDP-43 protein according to claim 1, wherein the amino acid sequence of the wild-type TDP-43 protein is as set forth in SEQ ID No: 2, 4, 6 or 8.

3. DNA encoding the mutant TDP-43 protein according to claim 1.

4. A vector comprising the DNA according to claim 3.

5. The vector according to claim 4, which is an adeno-associated virus vector.

6. An animal model for mutant TDP-43 protein accumulation, in which the mutant TDP-43 protein according to claim 1 is expressed in the brain of a non-human mammal.

7. A cell model for mutant TDP-43 protein accumulation, in which an aggregate of the mutant TDP-43 protein according to claim 1 is introduced into a cell.

8. The cell model according to claim 7, wherein the aggregate of the mutant TDP-43 protein is derived from the brain of an animal model for mutant TDP-43 protein accumulation, in which the mutant TDP-43 protein is expressed in the brain of a non-human mammal.

9. The cell model according to claim 8, wherein the aggregate of the mutant TDP-43 protein has seeding activity by which it functions as a seed for intracellular accumulation of the protein.

10. A method of screening for a therapeutic drug for neurodegenerative disease, comprising contacting or administering a test candidate substance to the animal model according to claim 6.

11. A kit of screening for a therapeutic drug for neurodegenerative disease, including the animal model according to claim 6.

12. A method of screening for a therapeutic drug for neurodegenerative disease, comprising contacting or administering a test candidate substance to the cell model according to claim 7.

13. A kit of screening for a therapeutic drug for neurodegenerative disease, including the cell model according to claim 7.


SEQUENCE LISTING (20 sequences)

SEQ ID NO: 1 - DNA, 1245 bp, Homo sapiens
  source 1..1245: mol_type = genomic DNA
atgtctgaatatattcgggtaaccgaagatgagaacgatgagcccattgaaataccatcggaagacgatgggacggtgctgctctccacggttacagcccagtttccaggggcgtgtgggcttcgctacaggaatccagtgtctcagtgtatgagaggtgtccggctggtagaaggaattctgcatgccccagatgctggctggggaaatctggtgtatgttgtcaactatccaaaagataacaaaagaaaaatggatgagacagatgcttcatcagcagtgaaagtgaaaagagcagtccagaaaacatccgatttaatagtgttgggtctcccatggaaaacaaccgaacaggacctgaaagagtattttagtacctttggagaagttcttatggtgcaggtcaagaaagatcttaagactggtcattcaaaggggtttggctttgttcgttttacggaatatgaaacacaagtgaaagtaatgtcacagcgacatatgatagatggacgatggtgtgactgcaaacttcctaattctaagcaaagccaagatgagcctttgagaagcagaaaagtgtttgtggggcgctgtacagaggacatgactgaggatgagctgcgggagttcttctctcagtacggggatgtgatggatgtcttcatccccaagccattcagggcctttgcctttgttacatttgcagatgatcagattgcgcagtctctttgtggagaggacttgatcattaaaggaatcagcgttcatatatccaatgccgaacctaagcacaatagcaatagacagttagaaagaagtggaagatttggtggtaatccaggtggctttgggaatcagggtggatttggtaatagcagagggggtggagctggtttgggaaacaatcaaggtagtaatatgggtggtgggatgaactttggtgcgttcagcattaatccagccatgatggctgccgcccaggcagcactacagagcagttggggtatgatgggcatgttagccagccagcagaaccagtcaggcccatcgggtaataaccaaaaccaaggcaacatgcagagggagccaaaccaggccttcggttctggaaataactcttatagtggctctaattctggtgcagcaattggttggggatcagcatccaatgcagggtcgggcagtggttttaatggaggctttggctcaagcatggattctaagtcttctggctggggaatgtag

SEQ ID NO: 2 - protein, 414 aa, Homo sapiens
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 3 - DNA, 1245 bp, Mus musculus
  source 1..1245: mol_type = genomic DNA
atgtctgaatatattcgggtaacagaagatgagaacgatgaacccattgaaataccatcagaagacgatgggacggtgttgctgtccacagttacagcccagtttccaggggcatgcggcctgcgctaccggaatcccgtgtctcagtgtatgagaggagtccgactggtggaaggaattctgcatgccccagatgctggctggggcaatctggtatatgttgtcaactatcccaaagataacaaaaggaaaatggatgagacagatgcttcctctgcagtgaaagtgaaaagagcagtccagaaaacatctgacctcatagtgttgggtctcccctggaaaacaactgagcaggatctgaaagactatttcagtacttttggagaggttcttatggttcaggtcaagaaagatcttaaaactggtcactcgaaagggtttggctttgttcgatttacagaatatgaaacccaagtgaaagtaatgtcacaacgacatatgatagatgggcgatggtgtgactgtaaacttcccaactctaagcaaagcccagacgagcctttgagaagcagaaaggtgtttgttggacgttgtacagaggacatgactgctgaagagcttcagcagtttttctgtcagtatggagaagtggtagatgtcttcattcccaaaccattcagagcttttgccttcgtcacctttgcagatgataaggttgcccagtctctttgtggagaggatttgatcattaaaggaatcagcgtgcatatatccaatgctgaacctaagcataatagcaatagacagttagaaagaagtggaagatttggtggtaatccaggtggctttgggaatcagggtgggtttggtaacagtagagggggtggagctggcttgggaaataaccagggtggtaatatgggtggagggatgaactttggtgcttttagcattaacccagcgatgatggctgcggctcaggcagcgttgcagagcagttggggtatgatgggcatgttagccagccagcagaaccagtcgggcccatctgggaataaccaaagccagggcagcatgcagagggaaccaaatcaggcttttggttctggaaataattcctacagtggttctaattctggtgccccccttggttgggggtcagcatcaaatgcaggatcgggcagtggttttaatgggggctttggctcgagcatggattctaagtcttctggctggggaatgtag

SEQ ID NO: 4 - protein, 414 aa, Mus musculus
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKDYFSTFGEVLMVQVKKDLKTGHSKGFGFVRFTEYETQVKVMSQRHMIDGRWCDCKLPNSKQSPDEPLRSRKVFVGRCTEDMTAEELQQFFCQYGEVVDVFIPKPFRAFAFVTFADDKVAQSLCGEDLIIKGISVHISNAEPKHNSNRQLERSGRFGGNPGGFGNQGGFGNSRGGGAGLGNNQGGNMGGGMNFGAFSINPAMMAAAQAALQSSWGMMGMLASQQNQSGPSGNNQSQGSMQREPNQAFGSGNNSYSGSNSGAPLGWGSASNAGSGSGFNGGFGSSMDSKSSGWGM

SEQ ID NO: 5 - DNA, 1245 bp, Rattus norvegicus
  source 1..1245: mol_type = genomic DNA
atgtctgaatatattcgggtaacagaagatgagaatgatgagcccattgaaataccatcagaagacgatgggacagtgttgctgtccacagttacagcccagtttccaggggcgtgtggcctgcgctaccggaatccagtgtctcagtgtatgagaggtgtccgactggtggaaggaattctgcatgccccagatgctggctggggcaatctggtctatgttgtcaactatcccaaagataacaaaaggaaaatggatgaggcggatgcttcctctgcagtgaaagtgaaaagagcagtccagaagacatctgacctcatagtgttgggtctcccctggaaaacaacagagcaggacctaaaagactacttcagtacttttggagaggttcttatggttcaggtcaagaaagatcttaaaactggtcactcaaaagggtttggctttgttcgatttacggaatatgaaactcaagtgaaagtaatgtcacagcgacatatgatagatgggcgatggtgtgactgtaaacttccaaattctaagcaaagcccagacgagcctttgagaagcagaaaggtgtttgttggacgttgtacagaggacatgactgctgaagagcttcagcagttcttctgtcagtatggagaagtggtagatgtcttcattcccaaaccattcagagcttttgcctttgttacctttgcagatgataaggttgcccagtctctttgtggagaggacttgatcattaaaggaatcagcgtgcatatatccaatgctgaacctaagcataatagcaatagacagttagaaagaagtggaagatttggtggtaatccaggtggctttgggaatcagggtgggtttggtaacagtagagggggtggagctggcttgggaaataaccagggtggtaatatgggtggagggatgaattttggtgcttttagcattaatccagcgatgatggctgcagctcaggcagcgttacagagcagttggggtatgatgggcatgttagccagccagcagaaccagtcaggcccatctgggaataaccaaagccagggcagcatgcagagggaaccaaatcaggcttttggttctggaaataactcttacagtggttctaattctggtgccccccttggttgggggtcagcatcaaatgcaggatcaggcagtggttttaatggaggctttggctcaagcatggattctaaatcctctggctggggaatgtag

SEQ ID NO: 6 - protein, 414 aa, Rattus norvegicus
MSEYIRVTEDENDEPIEIPSEDDGTVLLSTVTAQFPGACGLRYRNPVSQCMRGVRLVEGILHAPDAGWGNLVYVVNYPKDNKRKMDEADASSAVKVKRAVQKTSDLIVLGLPWKTTEQDLKDYFSTFGEVLMVQVKKDLKTGHSKGFGFVRFTEYETQVKVMSQRHMIDGRWCDCKLPNSKQSPDEPLRSRKVFVGRCTEDMTAEELQQFFCQYGEVVDVFIPKPFRAFAFVTFADDKVAQSLCGEDLIIKGISVHISNAEPKHNSNRQLERSGRFGGNPGGFGNQGGFGNSRGGGAGLGNNQGGNMGGGMNFGAFSINPAMMAAAQAALQSSWGMMGMLASQQNQSGPSGNNQSQGSMQREPNQAFGSGNNSYSGSNSGAPLGWGSASNAGSGSGFNGGFGSSMDSKSSGWGM

SEQ ID NO: 7 - DNA, 1245 bp, Callithrix jacchus
  source 1..1245: mol_type = genomic DNA
atgtctgaatatattcgggtaaccgaagatgagaacgatgaacccattgaaataccatcggaagatgatgggaccgtgctgctgtccacggttacagcccagtttccaggggcctgtgggcttcgctacaggaatccagtgtctcaatgtatgagaggtgtccggctggtggaaggaattctgcatgcccccgatgctggctggggaaatctggtgtatgttgtcaactatcccaaagataacaaaagaaaaatggatgagacagatgcttcatcagcagtgaaagtgaaaagagcagtccagaaaacatccgatttaatagtgttgggtctcccatggaaaacaactgaacaggacctgaaagactattttagtacctttggagaagttcttatggtgcaggtcaagaaagatcttaagactggtcattcaaaggggtttggctttgttcgttttacggaatatgaaacacaggtgaaagtaatgtcacagcggcatatgatagatggacggtggtgtgactgtaaacttcctaattctaagcaaagtgcagatgagcctttgagaagcagaaaagtgtttgtggggcgctgtacagaggacatgactgaggacgagctgcggcagttcttctcccagtacggggaggtgatggatgtcttcatccccaagccgttcagggcctttgcctttgtaacatttgcagatgatcagattgcgcaatctctttgtggcgaggacttgatcattaaaggaatcagcgttcacatatccaatgccgaacctaagcacaatagcaatagacagttagaaagaagtggaagatttggtggtaatccaggtggctttgggaatcagggtggatttggtaatagtagagggggtggagctggtttgggaaacaatcaaggtagtaatatgggtggtgggatgaactttggcgcattcagcattaatccagccatgatggctgcagcccaggcagcgctacagagcagttggggtatgatgggcatgttagccagccagcagaaccagtcaggcccatcgggtaataaccaaagccaaggcaacatgcagagggagccaaaccaggcctttggttctggaaataactcttatagtggttctaattctggtgcagcaattggttggggatcagcatcaaatgcagggtcgggtagtggttttaatggaggctttggctcaagcatggattctaagtcttctggctggggaatgtag

SEQ ID NO: 8 - protein, 414 aa, Callithrix jacchus
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKDYFSTFGEVLMVQVKKDLKTGHSKGFGFVRFTEYETQVKVMSQRHMIDGRWCDCKLPNSKQSADEPLRSRKVFVGRCTEDMTEDELRQFFSQYGEVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQSQGNMQREPNQAFGSGNNSYSGSNSGAAIGWGSASNAGSGSGFNGGFGSSMDSKSSGWGM

SEQ ID NO: 9 - DNA, 33 bp, synthetic construct
  source 1..33: mol_type = other DNA
gtatgttgtcaactatatggatgagacagatgc

SEQ ID NO: 10 - DNA, 33 bp, synthetic construct
  source 1..33: mol_type = other DNA
gcatctgtctcatccatatagttgacaacatac

SEQ ID NO: 11 - DNA, 49 bp, synthetic construct
  source 1..49: mol_type = other DNA
agactggtcattcaaaggggttgggcttggttcgttttacggaatatga

SEQ ID NO: 12 - DNA, 49 bp, synthetic construct
  source 1..49: mol_type = other DNA
tcatattccgtaaaacgaaccaagcccaacccctttgaatgaccagtct

SEQ ID NO: 13 - DNA, 43 bp, synthetic construct
  source 1..43: mol_type = other DNA
ctttgagaagcagaaaagtgttggtggggcgctgtacagagga

SEQ ID NO: 14 - DNA, 43 bp, synthetic construct
  source 1..43: mol_type = other DNA
tcctctgtacagcgccccaccaacacttttctgcttctcaaag

SEQ ID NO: 15 - DNA, 49 bp, synthetic construct
  source 1..49: mol_type = other DNA
tccccaagccattcagggccttggccttggttacatttgcagatgatca

SEQ ID NO: 16 - DNA, 49 bp, synthetic construct
  source 1..49: mol_type = other DNA
tgatcatctgcaaatgtaaccaaggccaaggccctgaatggcttgggga

SEQ ID NO: 17 - DNA, 36 bp, synthetic construct
  source 1..36: mol_type = other DNA
ggatccgccgccaccatgtctgaatatattcgggta

SEQ ID NO: 18 - DNA, 33 bp, synthetic construct
  source 1..33: mol_type = other DNA
cgcggccgcactagtctacattccccagccaga

SEQ ID NO: 19 - DNA, 18 bp, synthetic construct
  source 1..18: mol_type = other DNA
tggcgtggtgtgcactgt

SEQ ID NO: 20 - DNA, 20 bp, synthetic construct
  source 1..20: mol_type = other DNA
agggacgtagcagaaggacg